(19) United States
(12) Patent Application Publication
(10) Pub. No.: US 2005/0070886 A1
(43) Pub. Date: Mar. 31, 2005

(54) Method for treatment of varices

(75) Inventors: Franco Maglione, Napoli (IT); Tiziano Caldera, Cambs (GB); Fabio Coluccia, Milano (IT)

Correspondence Address: BOLESH J. SKUTNIK PhD. JD, 515 Shaker Road, East Longmeadow, MA, 01028, US

(73) Assignee: CeramOptec Industries, Inc.

(21) Appl. No.: 10/675,461

(22) Filed: Sep. 30, 2003

(51) Int. Cl. A61B 18/18

(52) U.S. Cl. 606/15

(57) ABSTRACT

A minimally invasive method for treating varices including pelvic varices in females, varicoceles, and also oesophageal varices is disclosed. The method comprises the steps of inserting a catheter device into the blood vessels of a patient and advancing the distal end of the catheter to reach the varix or varices. The insertion may be made in the femoral vein or in other vessels as appropriate. Preferably, x-ray, angiography, or other imaging techniques are used to visualize and position the catheter. An optical fiber or optical fiber bundle is then inserted into the catheter and the distal end is advanced to a predetermined point near the varix or varices. Laser energy of preferably 980 nm is then transmitted to the varix to close the blood vessel. Imaging techniques such as angiographies may again be performed to confirm closure of the vein. The method is an out-patient procedure that requires no incision or general anesthesia, requires no recovery time, and does not require that any foreign objects be left in the body. This method has been shown to have a higher success rate than previous embolization and surgical procedures.

### BACKGROUND OF THE INVENTION

1. Field of the invention

The invention relates to the field of treatment for male and female reproductive varices, in particular for varicoceles in men and pelvic varices in women, and including oesophageal varices..

2. Information Disclosure Statement

Varices are uneven, permanent dilations of veins than can occur in numerous areas of the body, and are very common in the superficial veins of the lower limbs (varicose, spider veins). One manifestation of these varices that occurs in men, and is the most common cause of male infertility, is the varicocele. Between 10 and 20% of post-pubescent males are affected by varicoceles, and 20-40% of all infertile males have varicoceles. A varicocele is a mass of enlarged veins in the spermatic cord, which extends from the testes up through the inguinal canal in the lower abdominal wall to the circulatory system. A varicocele can develop in one or both testicles, but in most cases occurs in the left testicle. Because of the varicocele's impairment of blood flow, the blood cannot cool as it does in normal veins, and this increased temperature is thought to be a cause of infertility. The excess heat can damage sperm or impede the production of new healthy sperm.

Varices can also play a role in chronic pelvic pain syndrome in women. One of numerous causes of chronic pelvic pain in women is “pelvic congestion syndrome” (PCS), a condition in which varices form in the pelvis minor, affecting organs including the uterus, ovaries and vulva. PCS is analogous to varicoceles in men. Pelvic varices occur in around 10% of the female population, and varices occur usually in women of the ages of 20-50.

A variety of treatments have been attempted for female pelvic varices, with varying degrees of success. Medications, including vasoconstrictors and hormonal medications, have been used successfully, though they may not always provide long-term relief. Numerous surgical treatments have also been performed, including tying off or removing veins, uterine suspension, and, generally as a last resort, hysterectomy.

Numerous options are also available for treatment of male varicoceles. Surgical treatment, usually performed under general anesthetic, involves making an incision above the scrotum and tying off the veins to detour blood flow into normal veins. Such a procedure can require up to six weeks recovery time before heavy lifting can be performed, with light activities able to be performed earlier.

Embolization is a procedure used by surgeons to block fluid flow through a blood vessel or organ, and has been used to treat both male varicoceles and female pelvic varices with at least some success. An embolus, which is a mass of some material, is inserted into the blood vessel with a catheter and is lodged in the vessel to restrict blood or fluid flow. This causes a clot to develop in the vessel that closes off the vessel. Types of emboli include wire coils, sponges, “chemical cross-linking means such as cyanoacrylate”, balloons, umbrella-like devices and other types of plugs. (U.S. Pat. No. 5,167,624)

U.S. Pat. No. 4,509,504 discloses the use of a device consisting of a material that swells when in contact with body fluid. The device is inserted into a body passage and the material is swelled to occlude the passage. The passages could be blood vessels (including varicose veins), urethers, spermatic ducts and oviducts. The device can purportedly be used as a contraceptive. When swelled, the device anchors in position and fully occludes the passage.

U.S. Pat. No. 6,200,332 describes a device and method for underskin laser treatment. Indications such as wrinkles and varicose veins can be treated with this invention. The handpiece uses a standard needle to insert an optical fiber under the skin or into a blood vessel, and features an extension piece that maintains the end of the optical fiber in a fixed position relative to the handpiece. The method described in this invention is generally envisioned for the treatment of tissue near the surface of the skin, for cosmetic procedures such as surface varicose veins or wrinkle removal. It may not be suitable for deep interior treatments such as varicoceles or pelvic varices

U.S. Pat. No. 5,167,624 describes a method and apparatus for passing an embolus into a blood vessel. The embolus lodges in the vessel and allows formation of a vessel-occluding clot around the embolus. The embolus is hydraulically passed through the lumen of a catheter to a given point in a blood vessel, the hydraulic fluid pushed the embolus to a predetermined position in the blood vessel. The embolus is preferably a coil that is stretched in the catheter lumen and recoils once released into the blood vessels. The recoiling causes the spring to expand and exert a force on the wall of the blood lo vessel to anchor it in place.

U.S. application No. 2002/0156499 describes an apparatus and method featuring a deformable member for occluding a blood vessel. Upon application of force on the occluder, the occluder can be deformed to expand to fully occlude a vessel, and may further be anchored so that migration can be avoided.

WO 01/66016 A1 describes embolic particles, agents and compositions, visible by ultrasound, for embolization to treat various disorders such as varicocele. Microbubbles are incorporated into or around the particles to allow the particles or composition to visible by ultrasound, thus avoiding the need for fluoroscopy and contrast agents in angiography.

Embolization procedures are typically outpatient procedures that require 24 hours or less of recovery time. Disadvantages include the relative complexity of the inserted embolus and the associated delivery equipment and the increased risk of infection, migration, or other complications due to the need to deposit and secure a foreign body within the blood vessel.

There exists a need for a minimally invasive treatment of varicocele and other abnormal veins that does not require the permanent or extended insertion of foreign objects and can be performed with a minimum of pain and without significant recovery time. The present invention addresses this need.

### OBJECTIVES AND BRIEF SUMMARY OF THE INVENTION

It is an object of the present invention to provide an improved method for treatment of varices.

It is another object of the present invention to provide a method for treatment of varices, particularly pelvic varices, varicoceles and oesophageal varices that is minimally invasive, does not require general anesthesia, and requires little or no time for patient recovery.

It is still another object of the present invention to provide a method for treatment of pelvic varices, varicoceles, and oesophageal varices that does not require the deposition-of foreign objects in the body.

Briefly stated, the present invention discloses a minimally invasive method for treating varices including pelvic varices in females, varicoceles, and also oesophageal varices. The method comprises the steps of inserting a catheter device into the blood vessels of a patient and advancing the distal end of the catheter to reach the varix or varices. The insertion may be made in the femoral vein or in other vessels as appropriate. Preferably, x-ray, angiography, or other imaging techniques are used to visualize and position the catheter. An optical fiber or optical fiber bundle is then inserted into the catheter and the distal end is advanced to a predetermined point near the varix or varices. Laser energy of preferably 980 nm is then transmitted to the varix to close the blood vessel. Imaging techniques such as angiographies may again be performed to confirm closure of the vein. The present invention is an out-patient procedure that requires no incision or general anesthesia, requires minimal recovery time, and does riot require that any foreign objects be left in the body. This method has been shown to have a higher success rate than previous embolization and surgical procedures.

The above, and other objects, features and advantages of the present invention will become apparent from the following description read in conjunction with the accompanying drawings.

### BRIEF DESCRIPTION OF FIGURES

FIG. 1—Illustration of the method for treatment of a varicocele.

### DETAILED DESCRIPTION OF PREFERRED EMBODIMENTS

The present invention is a method for treating male and female varices located in or near the reproductive system. In particular, the method is effective for treating varicoceles in men and pelvic varices in women. It is an outpatient procedure performed under local anesthetics, can be quickly performed with a minimum of recovery time, and avoids the need to introduce foreign objects into the body, in contrast to traditional embolization treatments.

In the first step, a small catheter is inserted into the circulatory system either at the groin, preferably into the femoral vein, or through an upper access such as the subclavian vein in the arm. Preferably under X-ray imaging, the catheter is moved into position near the varicocele vein mass. Optionally, further visualization procedures such as angiographies with contrast (dye), may be performed to achieve correct positioning

After the catheter is correctly positioned, a laser fiber is inserted into the catheter and secured to the catheter by a special locking system so that the distal end of the fiber extends a preselected distance from the distal part of the catheter itself. An exemplary embodiment of a locking system for use with the present invention is described in U.S. Pat. No. 6,200,332. That patent describes an underskin laser treatment device comprising a handpiece having a hollow channel and fitted with a hollow surgical needle through which an optical fiber, connected to a laser source, is inserted. The handpiece further comprises an extension that can fit into the hollow channel having a protrusion which is keyed to a groove within the channel wall for guiding the optical fiber through the handpiece and needle. This device, by virtue of the extension piece, maintains the optical fiber in a fixed position relative to, and at a fixed distance from, the handpiece, allowing the user to know how much of the fiber has been inserted into the treatment area. Laser energy having a wavelength of preferably 980 nm is then delivered following a preselected radiation protocol, which may vary by patient. Such protocol parameters include emitted power, duration of radiation or pulse, pulse length, and time between pulses.

Following irradiation, the catheter and fiber are withdrawn together until their ends reach the proximal part of the varicocele vein. The fiber is withdrawn through the catheter and dye may be injected so that further angiographies can be performed to confirm the closure of the vein. Other visualization techniques, such as echo color doppler ultrasound, may also be used.

Radiation is preferably delivered through an optical fiber or fiber bundle, and a preferred radiation source is a high power diode laser or diode array emitting at a wavelength of 980 nm. The optical fiber or bundle is connected to the radiation source at its proximal end and its distal end contains a means for distributing radiation. This radiation distribution means may take a variety of forms, including simply the bare end of the fiber or a shaped fiber tip, or including more complex means such as a diffuser. Laser energy, especially in the range of 980 nm, is effective at effecting vein closure by causing the vein wall to shrink and close off the vein. Damage to the vein wall creates a shrinking effect that acts to close off the vessel. The presence of blood in the vein plays a key role in evenly distributing thermal damage to the inner vein wall and creating damage over a wide inner surface area of the vein wall, and thus helps to more efficiently create a thrombotic occlusion and avoids simply cutting through the vein wall. Also, the radiation generates steam bubbles that serve to distribute a large amount of damaging thermal energy to large areas of the inner vessel wall. Another mechanism for closing the vein is the collapse of the vessel wall due to the 980 nm wavelength's high absorption in water. Alternatively, a large steam bubble may be generated by the radiation that forces blood from a section of the vein. This in turn makes it easier for the vessel to collapse. Additionally, the radiation can also have a coagulating effect on the blood, which can also aid in closing off or blocking the vein. These effects may act in conjunction to cause effective closure of the vein.

An advantage of the present invention is that a foreign body need not be inserted into the vein to trigger embolization. Because of this, there is no need for a mechanism to push the embolus into the vein, which simplifies both the procedure and the equipment needed. In addition, because no foreign body is left in the vein after treatment, the present invention does not introduce the risk of infection that is present in traditional embolization techniques. Also, the risk of migration, inherent in many detached occluding devices, is eliminated with the present invention.

There are numerous additional advantages of the present invention over prior art treatments. The method of the present invention does not require more than a small incision (large enough to insert the small catheter), resulting in faster recovery and a lack of scars. The use of general anesthetics can be avoided. Early data (a few cases) show higher success rate then embolization. Lastly, little or no recovery time is required and patients can resume activities faster than after surgery.

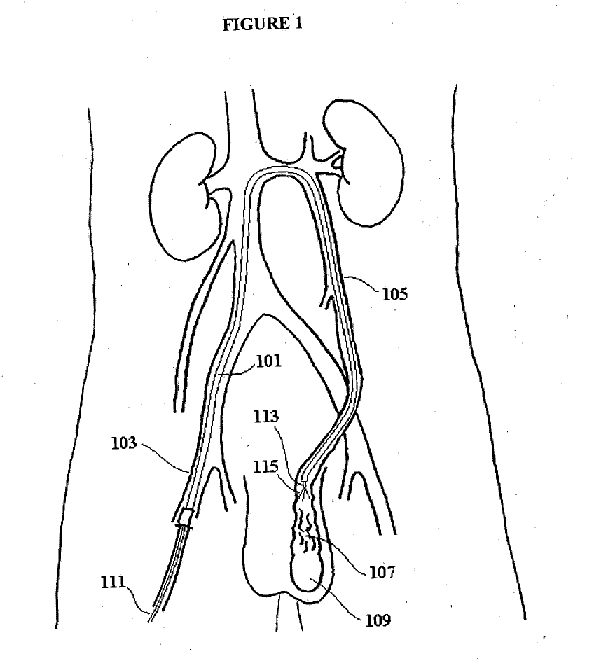

A preferred embodiment of the present invention is illustrated in FIG. 1. In this embodiment, a varicocele above the left testicle is closed using radiation. Catheter 101 is inserted through the front of a patient's leg into right femoral vein 103. The distal end of catheter 101 is advanced through right femoral vein 103 and through left internal spermatic vein 105 to a position near varicocele 107 above left testicle 109. Next, fiber 111 is inserted into catheter 101 and advanced until distal end 113 of fiber 111 has advanced a predetermined distance from the distal end of catheter 101. A radiation source is then activated and radiation 115 closes off the veins of varicocele 107. Fiber 113 is then withdrawn and catheter 101 may be withdrawn or used in an angiography to confirm that the varicocele has been effectively closed.

The present invention is not merely limited to varicocele and female pelvic varices treatment. Oesophageal varices may also be treated successfully using the method of the present invention. Oesophageal varices are enlarged veins on the lining of the esophagus that are prone to bleeding. They are life-threatening, and can be fatal in up to 50% of patients. They usually appear in patients with severe liver disease. For treatment of this indication, the method of the present invention is similar to that described above for varicoceles and female pelvic varices. In this embodiment, the catheter is inserted into the portal vein and advanced to a point proximate to the varices. Radiation as indicated above is applied to close off the vein.

The present invention is further illustrated by the following example, but is not limited thereby.

### EXAMPLE 1

### Treatment of a Varicocele Above the Left Testicle

The present invention is very effective for treating varicoceles in the spermatic cord above the testicle. In one example, in this case for a varicocele above the left testicle of a patient, a catheter is inserted into the right femoral vein and advanced so that the distal end of the catheter is approximately 1 cm from the site the desired site of embolization. X-ray imaging and angiography are performed to visualize this step and aid insertion.

A 400 micron optical fiber is coupled to a 980 nm diode laser and the fiber is advanced through the catheter until its distal end extends 1 cm from the distal end of the catheter. The fiber/catheter is fitted with a locking mechanism so that the fiber can be properly extended without further visualization or measurement.

The diode laser is activated with a power of 4-5 W, and radiation is applied in a series of pulses. Each pulse length is between 1 and 1.5 seconds long, separated by a span of 1 second between each pulse. Total irradiation time, or total time in which the laser is lion”, is between 15-30 seconds, depending on the length of the vein treated.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A method for the treatment of varices, comprising the steps of:
a. inserting a catheter into a blood vessel and advancing a distal end of said catheter to a position proximate to at least one varix;
b. inserting an optical waveguide, whose proximal end is connected to a radiation source and whose distal end comprises a radiation distribution means, into said catheter;
c. advancing said distal end of said waveguide through said distal end of said catheter to a predetermined point near said at least one varix;
d. irradiating said varix with radiation from said radiation source so as to cause closure of said at least one varix.

2. The method for treatment of varices according to claim 1, wherein said waveguide is selected from the group consisting of an optical fiber and an optical fiber bundle.

3. The method for treatment of varices according to claim 1, wherein said at least one varix is selected from the group consisting of a varicocele, a female pelvic varix, and an oesophageal varix.

4. The method for treatment of varices according to claim 1, wherein said radiation distribution device is selected from a group consisting of a bare fiber tip and a diffuser.

5. The method for treatment of varices according to claim 1, wherein said radiation source is selected from a group consisting of a diode laser and a diode laser array.

6. The method for treatment of varices according to claim 1, wherein said radiation has a wavelength of 980 nm.

7. The method for treatment of varices according to claim 1, wherein said predetermined point is 1 cm from said distal end of said catheter.

8. The method for treatment of varices according to claim 1, wherein said irradiation step is accomplished by employing a series of pulses.

9. The method for treatment of varices according to claim 1, comprising the additional step of utilizing x-ray and angiographic imaging to view a path of said catheter during said catheter insertion step.

10. The method for treatment of varices according to claim 1, comprising the additional step of utilizing echo color doppler ultrasound to view a path of said catheter during said catheter insertion step.

11. The method for treatment of varices according to claim 1, comprising the additional step of performing an additional angiography after said irradiation step to confirm closure of said varix.

12. The method for treatment of varices according to claim 2, wherein said optical fiber has a diameter of preferably 400 microns.
